(19) United States
(12) Patent Application Publication
(10) Pub. No.: US 2005/0186165 A1
(43) Pub. Date: Aug. 25, 2005

(54) Cosmetic composition based on a cosmetically active compound and a gel comprising at least one crosslinked network of crosslinked polymer particles

(76) Inventors: Estelle Mathonneau, Paris (FR); Isabelle Rollat, Paris (FR); Franck Giroud, Clichy (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/990,509

(22) Filed: Nov. 18, 2004

Related U.S. Application Data
(60) Provisional application No. 60/562,224, filed on Apr. 15, 2004.

(30) Foreign Application Priority Data
Nov. 18, 2003 (FR) 03 13448

(51) Int. Cl. A61K 7/09

(52) U.S. Cl. 424/70.2

(57) ABSTRACT

The present disclosure relates to a cosmetic composition, especially a hair composition, comprising, in a cosmetically acceptable medium, at least one cosmetically active compound and a gel comprising at least one crosslinked network of crosslinked polymer particles. The disclosure is also directed towards a method for fixing and/or holding a hairstyle using the composition, and also to the method of manufacturing a gel cosmetic formulation for holding and/or fixing the hairstyle.

This application claims benefit of U.S. Provisional Application No. 60/562,224, filed Apr. 15, 2004, which is incorporated by reference herein in its entirety.

The present disclosure relates to novel cosmetic compositions, including compositions for keratin fibers, such as hair, that are based on the combination of a cosmetically active compound and a gel comprising at least one crosslinked network of crosslinked polymer particles. The disclosure also relates to a process for treating keratin fibers, such as hair, and to a method of making these cosmetic compositions.

As used herein, the term “cosmetically active compound” means a product with a pleasant feel, aesthetic appearance, and pleasant odor, which may be used daily, for cosmetic purposes, and without a medical prescription for more than six months.

Styling products for forming locks of hair are generally gels containing a fixing polymer. But these gel styling products may have drawbacks: the hold of these locks in the desired shape may be limited over time and the fixing polymer incorporated into the gel may give the hair locks a tacky feel, which is cosmetically unappealing.

It is known practice to use a gel comprising at least one crosslinked network of crosslinked polymer particles, as described in patent application WO 03/022910. However, the gel-based compositions disclosed in WO 03/022910 comprise a pharmaceutical compound and are intended for medical application. No mention is made of combining the gel with a cosmetically active compound, for example, one that can be purchased over the counter.

Permanent-waving treatments for keratin fibers, and in particular the hair, are also known. These treatments include a reducing agent and an oxidizing agent, and require mechanical tensioning of the hair using rolling equipment in order to give the hair locks a long-lasting shape. Although they usually allow longer-lasting fixing of the hairstyle than gels combined with fixing polymers, these processes may degrade the feel quality of the hair.

The present inventors have discovered, surprisingly, that cosmetic compositions comprising, in a cosmetically acceptable medium, at least one cosmetically active compound and at least one gel comprising at least one crosslinked network of crosslinked polymer particles can give the hair good fixing and long-lasting cosmetic properties, and can improve the formation of rigid hair locks that are finer than those formed with the styling gels of the prior art, such as those styling gels containing a fixing polymer.

The present inventors have also found that the compositions disclosed herein can protect the surface of the hair and give it a pleasant feel. In addition, these compositions provide a coating to the hair.

Other aspects of the present disclosure will become apparent on reading the description and the examples that follow.

One aspect of the present disclosure is a cosmetic composition, such as a hair composition, comprising, in a cosmetically acceptable medium, at least one cosmetically active compound and at least one gel comprising at least one crosslinked network of crosslinked polymer particles.

Another aspect of the present disclosure is a method of making a cosmetic composition, such as a hair composition for holding and/or shaping the hairstyle, by including therein a gel comprising at least one crosslinked network of crosslinked polymer particles.

Another aspect of the present disclosure concerns a cosmetic hair process for fixing and/or holding a hairstyle using the cosmetic compositions disclosed herein.

The cosmetically active compound disclosed herein may be chosen from reducing agents or antioxidants, oxidizing agents, silicones, softeners, antifoams, moisturizers, emollients, plasticizers, sunscreens, dyes, pigments, fillers, clays, nacres or nacreous agents, fragrances, peptizers, thickeners, preserving agents, fixing polymers, vitamins or provitamins, conditioners such as oils, cationic polymers, cationic surfactants, waxes (including ceramides), and proteins.

Reducing agents may be chosen from organic or mineral reducing agents such as NaBH₄, KBH₄, BH₃, NADPH, NADH, H₂, 9-BBN (9 borabicyclo[3.3.1]nonane), LiAlH₄, NaAIEt₂H₂, Na₂S₂O₃, diisoamylborane, ascorbic acid, and cyclic or aliphatic compounds containing the enediol —(OH)C═C(OH)— chemical group (including the isomeric forms), and the salts and esters thereof, hydroquinone, NaBH(OAc)₃, formamidinesulfinic acid (FASA), N-phenylformadiminesulfinic acid, phosphines, NaBH₃CN, Na₂S₂O₄, BMS (bismercaptoethyl sulfone), DTT (dithiothreitol), DMH (N,N′-dimethyl-N,N′-bis(mercaptoacetyl)hydrazine), thiols, thiolates, citrates, sulfites, bisulfites, monopersulfates and bisulfates of alkali metals, enzymes of the protein disulfide isomerase or thioredoxine type, and sulfinates such as hydroxymethanesulfinates.

Oxidizing agents may be chosen from aqueous hydrogen peroxide solution, bromates, and persalts (e.g., persulfates and percarbonates).

Thickeners may be chosen from natural thickeners such as guar gum, xanthan gum and spruce meal, cellulose, and modified derivatives thereof, such as hydroxyethylcellulose, hydroxypropyl guar, or carboxyethylcellulose, and synthetic polymers of associative or non-associative nature, such as polymers based on acrylic monomers and polyurethane-type polycondensates. These thickeners may be of nonionic, anionic, cationic, or amphoteric nature.

Sunscreens may be chosen from anthranilates; cinnamic derivatives; dibenzoylmethane derivatives; salicylic derivatives; camphor derivatives; triazine derivatives (such as 1,3,5-triazine derivatives described in patent nos. U.S. Pat. No. 4,367,390, U.S. Pat. No. 4,724,137, EP 863 145, EP 517 104, EP 570 838, EP 796 851, EP 775 698, EP 878469, EP 933 376, EP 507 691, EP 507 692, EP 790 243 and EP 944 624); benzophenone derivatives; β,β′-diphenylacrylate derivatives; benzotriazole derivatives; benzalmalonate derivatives; benzimidazole derivatives; imidazolines; bis-benzazolyl derivatives (as described in EP 669 323 and U.S. Pat. No. 2,463,264); p-aminobenzoic acid (PABA) derivatives; methylenebis(hydroxyphenylbenzotriazole) derivatives (as described in patent nos. U.S. Pat. No. 5,237,071, U.S. Pat. No. 5,166,355, GB 2,303,546, DE 19 726 184 and EP 893 119); screening polymers and screening silicones (such as those described in patent application WO 93/04665); α-alkylstyrene-based dimers (such as those described in patent application DE 19 855 649); 4,4-diarylbutadienes (as described in patent applications EP 967 200 and DE 19 755 649); and mixtures thereof.

Among the dibenzoylmethane derivatives, 4-(tert-butyl)-4′-methoxydibenzoylmethane or Butyl Methoxy Dibenzoylmethane may be used, and these are sold under the trade name “Parsol® 1789” by the company Hoffmann LaRoche.

The hair dyes listed above may be direct dyes or oxidation dyes. The oxidation dyes that may be used herein are chosen from oxidation bases and/or couplers.

In some embodiments, the cosmetic compositions of the present disclosure comprise at least one oxidation base.

The oxidation bases that may be used herein are chosen from those conventionally known in oxidation dyeing. These include ortho- and para-phenylenediamines, double bases, ortho- and para-aminophenols, and heterocyclic bases, and also their acid-addition salts. The heterocyclic bases include pyridine derivatives, pyrimidine derivatives, pyrazole derivatives, and the acid-addition salts thereof.

The couplers that may be used as oxidation dyes include those conventionally used in oxidation dye compositions, e.g., meta-aminophenols, meta-phenylenediamines and meta-diphenols, naphthols and heterocyclic couplers such as indole derivatives, indoline derivatives, sesamol and its derivatives, pyridine derivatives, pyrazolotriazole derivatives, pyrazolones, indazoles, benzimidazoles, benzothiazoles, benzoxazoles, 1,3-benzodioxoles and quinolines, and the acid-addition salts thereof.

In general, the acid-addition salts of the oxidation bases and couplers may be chosen from the hydrochlorides, hydrobromides, sulfates, tartrates, lactates, and acetates.

The dyes used herein may also be chosen from natural and synthetic direct dyes. These direct dyes may then be chosen from neutral, cationic, or anionic nitro, azomethine, quinone and triarylmethane dyes, and plant extracts.

The pigments may be white or colored, mineral and/or organic, and coated or uncoated. Mineral pigments that may be used include titanium dioxide, optionally surface-treated, zirconium oxide, zinc oxide or cerium oxide, and also iron oxide, chromium oxide, manganese violet, ultramarine blue, chromium hydrate and ferric blue. Carbon black, pigments of D&C, and lakes based on cochineal carmine or on barium, strontium, calcium, or aluminium may be used as organic pigments. The nacres or nacreous agents may be chosen from white nacreous pigments such as mica coated with titanium or with bismuth oxychloride, colored nacreous pigments such as titanium mica with iron oxides, titanium mica containing only ferric blue or chromium oxide, titanium mica containing predominantly ferric blue or chromium oxide, titanium mica with an organic pigment listed above, nacreous pigments based on bismuth oxychloride, and insoluble organic ethers or esters.

As used herein, the term “fixing polymer” means any polymer, whether film-forming or not, that can give the hair a fixed shape or that can maintain this shape for a period of time. The fixing polymers that are suitable for use herein may be chosen from cationic, anionic, amphoteric, and nonionic polymers, and mixtures thereof.

The cationic fixing polymers may be chosen from polymers comprising primary, secondary, tertiary and/or quaternary amine groups forming part of the polymer chain or directly attached thereto. Additionally, these cationic fixing polymers have a number average molecular weight ranging from 500 to 5,000,000 and, in some embodiments, have a number average molecular weight ranging from 1,000 to 3,000,000.

Among these cationic fixing polymers, the following cationic polymer families may be used:

(1) homopolymers or copolymers derived from acrylic or methacrylic esters or amides with amine functions, which comprise at least one of the units of the following formula: 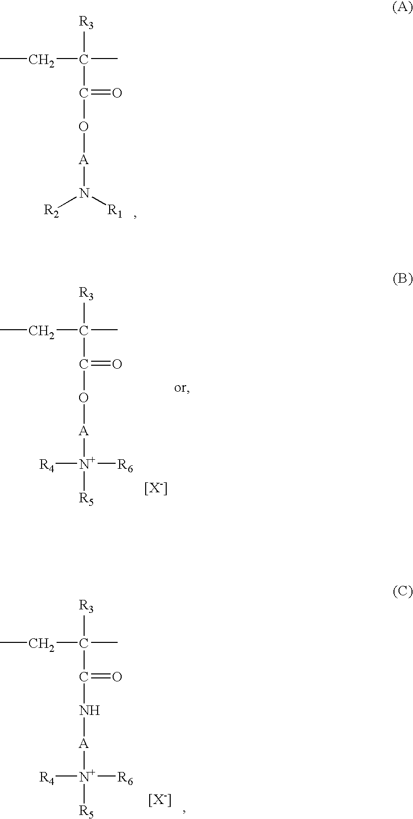
in which:
R₁ and R₂, which may be identical or different, are each chosen from a hydrogen atom and alkyl groups having from 1 to 6 carbon atoms;
R₃ is chosen from a hydrogen atom and CH₃;
A is chosen from linear or branched alkyl groups comprising 1 to 6 carbon atoms and hydroxyalkyl groups comprising 1 to 4 carbon atoms;
R₄, R₅, and R₆, which may be identical or different, are chosen from alkyl groups having from 1 to 18 carbon atoms and from benzyl groups;
X is chosen from a methosulfate anion and a halide, such as chloride or bromide

The copolymers of family (1), listed above, may also contain one or more comonomer units that may be chosen from the family of acrylamides, methacrylamides, diacetone acrylamides, acrylamides, and methacrylamides substituted on the nitrogen with lower (C₁₋₄) alkyl groups, groups derived from acrylic or methacrylic acids or esters thereof, vinyllactams such as vinylpyrrolidone or vinylcaprolactam, and vinyl esters.

For example, copolymers of family (1) that may be used herein include:
copolymers of acrylamide and of dimethylaminoethyl methacrylate quaternized with dimethyl sulfate or with a dimethyl halide, such as the one sold under the name Hercofloc® by the company Hercules;
copolymers of acrylamide and of methacryloyloxyethyltrimethylammonium chloride, described, for example, in patent application EP-A-080 976 and sold under the name Bina Quat P100 by the company Ciba Geigy;
copolymers of acrylamide and of methacryloyloxyethyltrimethylammonium methosulfate, such as the product sold under the name Reten® by the company Hercules;
quaternized or non-quaternized vinylpyrrolidone/dialkylaminoalkyl acrylate or methacrylate copolymers, such as the products sold under the name Gafqua® by the company ISP (e.g., Gafquat® 734 or Gafquate 755), or alternatively, the products known as Copolymer® 845, 958, and 937 (these polymers are described in detail in French Patent Nos. 2,077,143 and 2,393,573);
fatty-chain polymers containing a vinylpyrrolidone unit, such as the products sold under the name Styleze W20 and Styleze W10 by the company ISP;
dimethylaminoethyl methacrylate/vinylcaprolactam/vinylpyrrolidone terpolymers, such as the product sold under the name Gaffix® VC 713 by the company ISP; and
quaternized vinylpyrrolidone/dimethylaminopropylmethacrylamide copolymers, such as the product sold under the name Gafquat® HS 100 by the company ISP;
(2) cationic polysaccharides, for instance, those containing quaternary ammonium, such as those described in U.S. Pat. Nos. 3,589,578 and 4,031,307, such as guar gums containing trialkylammonium cationic groups. Such products are sold, for example, under the trade names Jaguar C13S, Jaguar C15 and Jaguar C17 by the company Meyhall;
(3) quaternary copolymers of vinylpyrrolidone and of vinylimidazole;
(4) chitosans or salts thereof, wherein the salts that can be used include chitosan acetate, lactate, glutamate, gluconate or pyrrolidonecarboxylate. Among these compounds, mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol; and
(5) cationic cellulose derivatives such as copolymers of cellulose or of cellulose derivatives grafted with a water-soluble monomer comprising a quaternary ammonium, which is disclosed in U.S. Pat. No. 4,131,576. Such cationic cellulose derivatives include hydroxyalkylcelluloses, for instance hydroxymethyl-, hydroxyethyl- or hydroxypropylcelluloses grafted with a methacryloyloxyethyltrimethylammonium, methacrylamidopropyltrimethylammonium, or dimethyldiallylammonium salt. Products corresponding to this definition are sold under the name Celquat L 200 and Celquat H 100 by the company National Starch.

In addition, the cationic fixing polymers may also be chosen from cationic grafted silicone polymers.

The anionic fixing polymers used herein are polymers comprising groups derived from carboxylic acid, sulfonic acid, or phosphoric acid and have a number average molecular weight ranging from 500 to 5,000,000.

The carboxylic groups are provided by unsaturated monocarboxylic or dicarboxylic acid monomers, such as those corresponding to the formula: 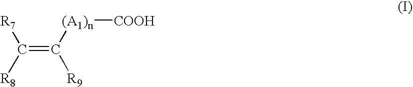
in which:
n is an integer from 0 to 10;
A₁ is a methylene group, optionally connected to the carbon atom of the unsaturated group, or to the neighboring methylene group when n is greater than 1, via a hetero atom such as oxygen or sulfur;
R₇ is chosen from a hydrogen atom, a phenyl group, and a benzyl group;
R₈ is chosen from a hydrogen atom and a lower alkyl or carboxyl group; and
R₉ is chosen from a hydrogen atom, a lower alkyl group, a —CH₂—COOH group, a phenyl group, and a benzyl group.

In one embodiment of the above-mentioned formula, the term “lower alkyl group” means a group having 1 to 4 carbon atoms, and in some embodiments, the lower alkyl group may be methyl or ethyl.

Anionic fixing polymers containing carboxylic groups that may be used herein include:
(1) acrylic or methacrylic acid homo- or copolymers, or salts thereof such as the products sold under the names Versicol® E or Versicol® K by the company Allied Colloid and Ultrahold® by the company BASF, and the copolymers of acrylic acid and of acrylamide sold in the form of their sodium salts (the sodium salts of polyhydroxycarboxylic acids) under the names Reten® 421, 423 or 425 by the company Hercules; and
(2) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in French Patent No. 1,222,944 and German Patent Application 2,330,956. The copolymers of this type, which comprise an optionally N-alkylated and/or hydroxyalkylated acrylamide unit in their chain, are described in Luxembourg Patent Applications 75,370 and 75,371 and are sold under the name Quadramer® by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, e.g., copolymers of lauryl, sold by the company ISP under the name Acrylidone® LM and methacrylic acid/ethyl acrylate/tert-butyl acrylate terpolymers such as the product Luvimer® 100 P is sold by the company BASF. Mention may also be made of methacrylic acid/acrylic acid/ethyl acrylate/methyl methacrylate copolymers as an aqueous dispersion sold under the name Amerhold® DR 25 by the company Amerchol;
(3) crotonic acid copolymers, such as those comprising vinyl acetate or propionate units in their chain and optionally other monomers such as allylic esters or methallylic esters, vinyl ether or vinyl ester of a linear or branched saturated carboxylic acid with a long hydrocarbon chain (such as those containing at least 5 carbon atoms), it being possible for these polymers to be grafted or crosslinked, or alternatively, another vinyl, allylic or methallylic ester monomer of an α- or β-cyclic carboxylic acid. Such polymers are described, inter alia, in French Patent Nos. 1,222,944, 1,580,545, 2,265,782, 2,265,781, 1,564,110, and 2,439,798. Commercial products falling into this class include the 28-29-30, 26-13-14, and 28-13-10 resins sold by the company National Starch;
(4) copolymers derived from C₄-C₈ monounsaturated carboxylic acids or anhydrides chosen from:
copolymers comprising (i) one or more maleic, fumaric, or itaconic acids or anhydrides; and (ii) at least one monomer chosen from vinyl esters, vinyl ethers, vinyl halides, phenylvinyl derivatives, acrylic acid and its esters, wherein the anhydride functions of these copolymers are optionally monoesterified or monoamidated. Such polymers are described in U.S. Pat. Nos. 2,047,398, 2,723,248, and 2,102,133 and GB Patent No. 839,805, and are sold under the names Gantrez® AN or Gantreze ES by the company ISP; and
copolymers comprising (i) one or more maleic, citraconic or itaconic anhydride units; and (ii) one or more monomers chosen from allylic or methallylic esters optionally comprising one or more acrylamide, methacrylamide, α-olefin, acrylic or methacrylic ester, acrylic or methacrylic acid or vinylpyrrolidone groups in their chain, wherein the anhydride functions of these copolymers are optionally monoesterified or monoamidated. These polymers are described, for example, in French Patent Nos. 2,350,384 and 2,357,241.

The homopolymers and copolymers comprising sulfonic groups are polymers comprising vinylsulfonic, styrenesulfonic, naphthalenesulfonic or acrylamidoalkylsulfonic units.

By way of example, these polymers can be chosen from:
polyvinylsulfonic acid salts having a molecular weight ranging from 1,000 to 100,000, as well as the copolymers with an unsaturated comonomer, such as acrylic or methacrylic acids and their esters, and acrylamide or its derivatives, vinyl ethers, and vinylpyrrolidone;
polystyrenesulfonic acid salts such as the sodium salts that are sold under the names Flexan®500 and Flexan® 130 by National Starch. These compounds are described in French Patent FR 2,198,719; and
polyacrylamidesulfonic acid salts, such as those mentioned in U.S. Pat. No. 4,128,631, for example, polyacrylamidoethylpropanesulfonic acid, sold under the name Cosmedia Polymer HSP 1180 by Henkel.

Additionally, the branched block anionic polymer sold under the name Fixate G100 by the company Noveon may be used as another anionic fixing polymer.

Further, the anionic fixing polymers may be chosen from acrylic acid copolymers, such as the acrylic acid/ethyl acrylate/N-tert-butylacrylamide terpolymers sold under the name Ultrahold® Strong by the company BASF; copolymers derived from crotonic acid, such as vinyl acetate/vinyl tert-butylbenzoate/crotonic acid terpolymers and the crotonic acid/vinyl acetate/vinyl neododecanoate terpolymers sold under the name Resin 28-29-30 by the company National Starch; polymers derived from maleic, fumaric, or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by the company ISP; the copolymers of methacrylic acid and of methyl methacrylate sold under the name Eudragit® L by the company Rohm Pharma; the copolymers of methacrylic acid and of ethyl acrylate sold under the name Luvimer® MAEX or MAE by the company BASF; the vinyl acetate/crotonic acid copolymers sold under the name Luviset CA 66 by the company BASF; the vinyl acetate/crotonic acid copolymers grafted with polyethylene glycol sold under the name Aristoflex A® by the company BASF; and the polymer sold under the name Fixate G100 by the company Noveon.

Among the anionic fixing polymers mentioned above, in some embodiments of the present disclosure, use may be made of the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone® LM by the company ISP, and the polymer sold under the name Fixate G100 by the company Noveon.

The amphoteric fixing polymers that can be used in accordance with the present disclosure can be chosen from polymers comprising units B and C distributed randomly in the polymer chain, in which B denotes a unit derived from a monomer comprising at least one basic nitrogen atom, and C denotes a unit derived from an acid monomer comprising one or more carboxylic or sulfonic groups. Alternatively, B and C can denote groups derived from carboxybetaine or sulfobetaine zwitterionic monomers or B and C can also denote a cationic polymer chain comprising primary, secondary, tertiary, or quaternary amine groups, in which at least one of the amine groups bears a carboxylic or sulfonic group connected via a hydrocarbon group. Additionally, in some embodiments, B and C form part of a chain of a polymer containing an α,β-dicarboxylic ethylene unit, in which one of the carboxylic groups has been made to react with a polyamine comprising one or more primary or secondary amine groups.

For instance, the amphoteric fixing polymers corresponding to the definition above may be chosen from the following polymers:

(1) copolymers having acidic vinyl and basic vinyl units, such as those resulting from the copolymerization of a monomer derived from a vinyl compound bearing a carboxylic group such as acrylic acid, methacrylic acid, maleic acid, α-chloroacrylic acid, and a basic monomer derived from a substituted vinyl compound containing at least one basic atom such as dialkylaminoalkyl methacrylate and acrylate, dialkylaminoalkylmethacrylamides, and -acrylamides. Such compounds are described in U.S. Pat. No. 3,836,537; and

(2) polymers comprising units derived from:
a) at least one monomer chosen from acrylamides and methacrylamides substituted on the nitrogen atom with an alkyl group;
b) at least one acidic comonomer containing one or more reactive carboxylic groups; and
c) at least one basic comonomer such as esters containing primary, secondary, tertiary, and quaternary amine substituents of acrylic and methacrylic acids and the product of quaternization of dimethylaminoethyl methacrylate with dimethyl or diethyl sulfate.

Some example N-substituted acrylamides or methacrylamides may be compounds in which the alkyl groups contain from 2 to 12 carbon atoms, e.g., N-ethylacrylamide, N-tert-butylacrylamide, N-tert-octylacrylamide, N-octylacrylamide, N-decylacrylamide, N-dodecylacrylamide, and the corresponding methacrylamides.

The acidic comonomers may be chosen from acrylic acid, methacrylic acid, crotonic acid, itaconic acid, maleic acid, and fumaric acid and alkyl monoesters, having 1 to 4 carbon atoms, of maleic or fumaric acids or anhydrides.

Some basic comonomers that may be used herein include aminoethyl, butylaminoethyl, N,N′-dimethylaminoethyl, and N-tert-butylaminoethyl methacrylates.

The copolymers whose CTFA (4th edition, 1991) name is octylacryl-amide/acrylates/butylaminoethyl methacrylate copolymer, such as the products sold under the name Amphomer® or Lovocryl® 47 by the company National Starch, may also be used.

(3) crosslinked and acylated polyamino amides partially or totally derived from polyamino amides of general formula: [symbol]
CCO—R₁₀—CO-Z [symbol] (II)
in which:
R₁₀ is a divalent group derived from a saturated dicarboxylic acid, a mono- or dicarboxylic aliphatic acid containing an ethylenic double bond, an ester of a lower alkanol, having 1 to 6 carbon atoms, of these acids, or a group derived from the addition of any one of said acids to a bis(primary) or bis(secondary) amine; and
Z is a group derived from a bis(primary), mono- or bis(secondary) polyalkylene-polyamine and optionally is chosen from:
   a) in proportions of from 60 to 100 mol %, the group:
      —NH [symbol] (CH₂)ₓ—NH [symbol] ₚ (III)
      wherein:
x=2 and p=2 or 3, or alternatively, x=3, and p=2, and where this group is derived from diethylenetriamine, from triethylenetetraamine, or from dipropylenetriamine;
   b) in proportions of from 0 to 40 mol %, the group (IV) above in which:
   x=2 and p=1, and which is derived from ethylenediamine, or the group derived from piperazine: 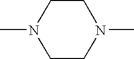
   c) in proportions of from 0 to 20 mol %, the —NH(CH₂)₆—NH— group being derived from hexamethylenediamine, these polyamino amides being crosslinked by addition reaction of a difunctional crosslinking agent chosen from epihalohydrins, diepoxides, dianhydrides, and bis-unsaturated derivatives, using from 0.025 to 0.35 mol of crosslinking agent per amine group of the polyamino amide, and acylated by the action of acrylic acid, chloroacetic acid, or an alkane sultone, or salts thereof.

The saturated carboxylic acids may be chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid, and itaconic acid.

The alkane sultones used in acylation may be propane sultone or butane sultone, and the salts of the acylating agents may be sodium or potassium salts.

(4) polymers comprising zwitterionic units of formula: 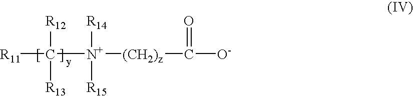
in which:
R₁ is a polymerizable unsaturated group such as an acrylate, methacrylate, acrylamide or methacrylamide group;
y and z are each independently an integer from 1 to 3;
R₁₂ and R₁₃ are each independently chosen from a hydrogen atom, and methyl, ethyl and propyl groups; and
R₁₄ and R₁₅ are each independently chosen from a hydrogen atom and an alkyl group, such that the sum of the carbon atoms in R₁₄ and R₁₅ does not exceed 10.

The polymers comprising such units can also comprise units derived from nonzwitterionic monomers such as dimethyl- or diethylaminoethyl acrylate or methacrylate or alkyl acrylates or methacrylates, acrylamides or methacrylamides or vinyl acetate.

By way of example, copolymers of methyl methacrylate/methyl dimethylcarboxymethylammonioethyl methacrylate, such as the product sold under the name Diaformer Z301 by the company Sandoz, may be used.

(5) polymers derived from chitosan comprising monomer units corresponding to the following formulae: 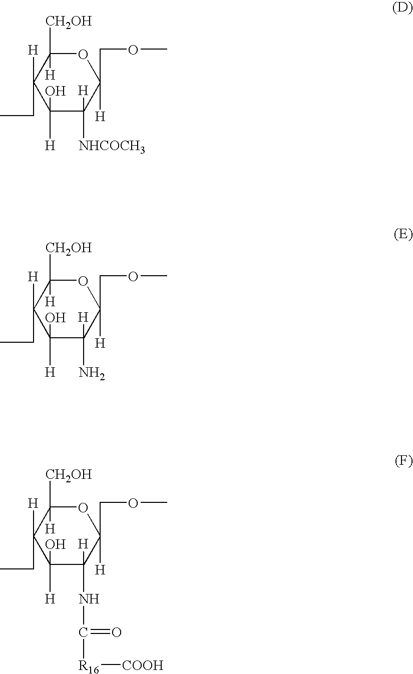
the unit (D) being present in proportions ranging from 0 to 30%, the unit (E) in proportions ranging from 5 to 50%, and the unit (F) in proportions ranging from 30% to 90%, where, in this unit (F), R₁₆ is a group of formula: 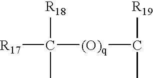
in which:
if q=0, R₁₇, R₁₈, and R₁₉, which may be identical or different, are each chosen from a hydrogen atom; methyl, hydroxyl, acetoxy, and amino groups; monoalkylamine and dialkylamine groups optionally interrupted by at least one nitrogen atom and/or optionally substituted with at least one group chosen from amine, hydroxyl, carboxyl, alkylthio and sulfonic groups; alkylthio groups in which the alkyl group bears an amino residue, at least one of the groups R₁₇, R₁₈ and R₁₉ being, in this case, a hydrogen atom; or
if q=1, R₁₇, R₁₈, and R₁₉, which may be identical or different, are each a hydrogen atom,
   as well as the salts formed by these compounds with bases or acids.

(6) polymers corresponding to the general formula (V) below, which are described, for example, in French Patent FR 1,400,366: 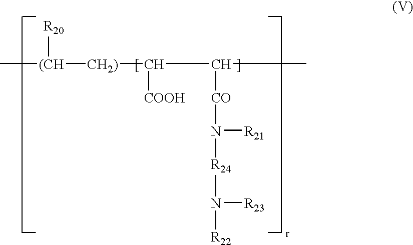
in which:
R₂₀ is chosen from a hydrogen atom, and CH₃O, CH₃CH₂O, and phenyl groups;
R₂₁ is chosen from a hydrogen atom and a lower alkyl group such as methyl or ethyl;
R₂₂ is chosen from a hydrogen atom and a C₁₋₆ lower alkyl group such as methyl or ethyl;
R₂₃ is chosen from a C₁₋₆ lower alkyl group such as methyl or ethyl and a group corresponding to the formula —R₂₄—N(R₂₂)₂, wherein R₂₂ is defined as above and R₂₄ is defined as below;
R₂₄ is chosen from —CH₂—CH₂—, —CH₂—CH₂—CH₂—, and —CH₂—CH(CH₃)— groups; and
r is such that the molecular weight of the polymer is from 500 to 6,000,000 or from 1,000 to 1,000,000.

(7) polymers derived from the N-carboxyalkylation of chitosan, such as N-carboxymethylchitosan or N-carboxybutylchitosan, sold under the name Evalsan by the company Jan Dekker;

(8) amphoteric polymers of the type -D-X-D-X—, chosen from:
a) polymers obtained by the action of chloroacetic acid or sodium chloroacetate on compounds comprising at least one unit of formula:
   -D-X-D-X-D- (VI)
   wherein:
D is a group 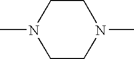
X is the symbol E or E′; and
E and E′, which may be identical or different, are chosen from divalent groups that are alkylene groups with a straight or branched chain containing up to 7 carbon atoms in the main chain, unsubstituted or substituted with hydroxyl groups and which can comprise, in addition to the oxygen, nitrogen and sulfur atoms, 1 to 3 aromatic and/or heterocyclic rings, wherein the oxygen, nitrogen, and sulfur atoms are present in the form of ether, thioether, sulfoxide, sulfone, sulfonium, alkylamine, or alkenylamine groups, hydroxyl, benzylamine, amine oxide, quaternary ammonium, amide, imide, alcohol, ester and/or urethane groups.
b) polymers of formula:
   -D-X-D-X— (VI′)
   wherein:
D is a group 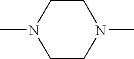
X is E or E′, and at least once E′;
E having the meaning given above; and
E′ is a divalent group that is an alkylene group with a straight or branched chain having up to 7 carbon atoms in the main chain, which is unsubstituted or substituted with at least one hydroxyl group and comprising at least one nitrogen atom, the nitrogen atom being substituted with an alkyl chain optionally interrupted by an oxygen atom and comprising one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric fixing polymers described above, the amphoteric fixing polymers of family (3), such as the copolymers whose CTFA name is octylacryl-amide/acrylates/butylaminoethyl methacrylate copolymer, such as the products sold under the names Amphomer®, Amphomer® LV 71, or Lovocryl® 47 by the company National Starch, and those of family (4), such as the copolymers of methyl methacrylate/methyl dimethylcarboxymethylammonioethyl methacrylate, sold, for example, under the name Diaformer® Z301 by the company Sandoz, may be used.

The nonionic fixing polymers that may be used according to the present disclosure are chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene; copolymers of vinyl acetate and of maleic ester, for example, of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer chosen, for example, from butadiene and alkyl (meth)acrylates, including the products sold under the name CJ 0601 B by the company Rohm & Haas;
styrene homopolymers;
styrene copolymers, for instance copolymers of styrene and of an alkyl (meth)acrylate, such as the products Mowilith® LDM 6911, Mowilith® DM 611, and Mowilith® LDM 6070 sold by the company Hoechst, the products Rhodopas® SD 215 and Rhodopas® DS 910 sold by the company Rhône-Poulenc; copolymers of styrene, of alkyl methacrylate and of alkyl acrylate, copolymers of styrene and of butadiene, or copolymers of styrene, of butadiene, and of vinylpyridine;
polyamides;
vinyllactam homopolymers other than vinylpyrrolidone homopolymers, such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37, and VA 28 by the company BASF, and poly(vinylpyrrolidone/vinyl acetate/vinyl propionate) terpolymers, for instance, the product sold under the name Luviskol® VAP 343 by the company BASF.

In certain embodiments, the alkyl groups of the nonionic polymers mentioned above contain from 1 to 6 carbon atoms.

According to the present disclosure, it is also possible to use fixing polymers of grafted silicone type comprising a polysiloxane portion and a portion comprising a nonsilicone organic chain, one of the two portions constituting the main chain of the polymer, and the other being grafted onto the main chain. These polymers are described, for example, in patent applications EP-A-412,704, EP-A-412,707, EP-A-640,105, and WO 95/00578, EP-A-582,152, and WO 93/23009 and patents U.S. Pat. No. 4,693,935, U.S. Pat. No. 4,728,571, and U.S. Pat. No. 4,972,037 and may be amphoteric, anionic, or non-ionic.

Such polymers are, for example, copolymers that can be obtained by free radical polymerization from the monomer mixture formed from:
a) 50 to 90% by weight of tert-butyl acrylate;
b) 0 to 40% by weight of acrylic acid; and
c) 5 to 40% by weight of a silicone macromer of formula: 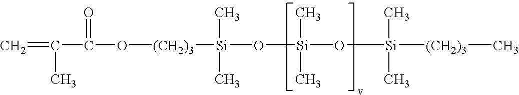
   in which v is a number from 5 to 700; the weight percentages being calculated relative to the total weight of the monomers.

Other examples of grafted silicone polymers are polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Another type of silicone fixing polymer is the product Luviflex® Silk sold by the company BASF.

Additionally, functionalized or non-functionalized, silicone or non-silicone, cationic, nonionic, anionic or amphoteric polyurethanes or mixtures thereof may also be used as fixing polymers.

The polyurethanes that may be used herein include those disclosed in patents EP 751,162, EP 637,600, EP 648,485 and FR 2,743,297, patents EP 656,021 or WO 94/03510 from the company BASF, and EP 619,111 from the company National Starch. Furthermore, the products sold under the names Luviset Pure and Luviset® Si-Pur by the company BASF are polyurethane products that may be used in the compositions disclosed herein.

The conditioners may be chosen from natural or synthetic, silicone or non-silicone, fluoro or non-fluoro, and linear or branched oils. Silicone oils that may be used include volatile or non-volatile, cyclic or non-cyclic, organomodified or non-organomodified oils. They may also be chosen from natural or synthetic, silicone or non-silicone, fluoro or non-fluoro waxes. In some embodiments, waxes of plant origin and ceramides may be used. The conditioners may also be chosen from cationic surfactants bearing at least one fatty chain and optionally comprising an ester function.

The conditioning polymers may be chosen from cationic polymers such as guar-based and cellulose-based cationic polymers and cyclopolymers comprising dimethyldialkylammonium chloride as monomer, and also amphoteric polymers and especially acrylic acid/dimethyldialkylammonium chloride copolymers.

The gel used in the compositions disclosed herein is a gel comprising at least one crosslinked network of crosslinked polymer particles. It is formed from interlinked polymer particles, and these particles are formed from crosslinked polymer. As used herein, the term “crosslinked polymer particles” means particles that comprise at least one crosslinked polymer. The gel may be formed by forming the polymer particles, crosslinking the polymer forming the particles, and then forming the crosslinked network of particles by crosslinking the particles together.

The polymer chains may be crosslinked together via functions present on the polymer or via crosslinking agents that may be chosen from divinyl ethers of aliphatic diols and divinyl ethers of aliphatic diols and divinyl ethers of polyethylene glycols, and also from 1,7-octadiene, 1,9-decadiene, divinylbenzene, N,N′-bis(methyleneacrylamide), polyethylene glycol diacrylate, polypropylene glycol diacrylate, trimethylolpropane triacrylate, and polyhydric alcohols esterified with an acrylic acid. In one embodiment, 1,9-decadiene is used.

The polymer chains may thus form particles of micrometric or nanometric size. In addition, the particles may be solid or hollow, and of spherical, elliptical, or polyhexagonal shape.

In one embodiment, the functions present on the polymer chains are sufficiently numerous to ensure the crosslinking of the polymer chains and to form the particles, and so that there can still be functions available at the surface of the particles for, in a second stage, crosslinking the particles together to form a network of particles.

In one embodiment, the polymer used to form the crosslinked network of crosslinked particles is a polymer that responds to an external stimulus. The term “polymer that responds to an external stimulus” means a polymer whose properties change reversibly when it is subjected to a stimulus such as temperature, UV rays, pH, an electrical field, a magnetic field, a change of solvent, or the degree of salinity. In one embodiment, the polymer is expected to shrink reversibly once subjected to one of these stimuli.

A polymer responding to an increase in temperature may be chosen from polyacrylamides, polyvinylcaprolactams, methyl polyvinyl ethers, 3-hydroxy polypropyl acrylates, polyvinyl acetates, polyvinyloxazolines, polyethylene oxides, polypropylene oxides, polyalkylacrylamides, polyhydroxypropylcelluloses and polyvinyl alcohols, and copolymers thereof. The polymers responding to a change in pH may be those cited in Y. Ito, S. Kotera, M. Inaba, K. Kono, Y. Imanishi, Polymer, 31, 2157-61 (1990), and Y. Ito, M. Inaba, D. J. Chung, Y. Imanishi, Macromolecules, 25, 7313-16 (1992). Polymers that change their conformation under the influence of UV rays may be chosen from polyacrylamides containing small amounts of triphenylmethane leucohydroxides or leucocyanide groups (see M. Irie, Applied Photochromic Polymer Systems., 174-206 (1992), M. Irie, Adv. Polym. Sci., 94, 27-67 (1990)). The polymers responding to changes of solvent may be chosen from those mentioned in references M. Ilavsky, J. Hronz, J. Stejskal, K. Boudal, Macromolecule, 17, 2668 (1984) and T. Tanaka, Sci. Am., 244,110 (1981) and, finally, the polymers responding to electrical fields may be chosen from those cited in T. Tanaka, I. Nishio, S. T. Sun, S. V. Nishio, Science, 218, 467 (1973).

By way of example, the following process may be performed: A polymer responding to temperature stimulus is dispersed in a solvent, and is heated to a temperature above the limit transition temperature for shrinkage such that the polymer chains shrink and form polymer particles. The polymer inside the particles is then crosslinked using functions present on the polymer or using at least one crosslinking agent. Finally, the polymer particles are crosslinked together, using a crosslinking agent, to form a crosslinked network of crosslinked polymer particles. In some embodiments, the crosslinked polymer particles may be formed from more than one polymer type.

In this network, the size of the particles may range from 1 to 100 nm, such as from 1 to 50 nm. The size of the particles in the network may be identical or different.

The gel contains at least one network of crosslinked particles. If the gel contains several networks, these networks will be interlaced. Each network may contain one or more types of polymer particles, and one or more types of polymers in each particle.

The crosslinked polymer particles may be present in an amount ranging from 0.4% to 60% based on the total weight of the composition, such as from 1% to 30%, for example, from 2% to 20% based on the total weight of the composition. The size of the network(s), i.e., the mean spacing between the crosslinked particles of the same network, may range from 50 to 1000 nm, for example, from 50 to 500 nm. The particles may be linked together via covalent bonds between functional groups located at the surface of the particles. Additionally, the crosslinking bonds between the particles are degradable or non-degradable and may be cleaved by an external or non-external stimulus.

The gel used in the compositions disclosed herein has a relatively stable conformation. The distance between the polymer particles is generally not perturbed by the addition of additives or the change of solvent or by mechanical stirring, in particular, for polymers responding to temperature and/or UV rays. Where the size and spacing between the particles are well-controlled (i.e., if the particles are monodisperse), and if the spacing between the particles is uniform (for polymers responding to an external stimulus), the network diffracts light and gives the gel an opal color. The color of the gel depends specifically on the spacing between the particles.

For a given polymer that may be used herein, the particle size depends on the type of polymer, the mode of synthesis, and the monomer/crosslinking agent ratio used for the synthesis process. The mean spacing between the various particles of the network depends on the mode of crosslinking used, and depends particularly on the proportion of polymer particles/crosslinking agents if crosslinking agents are used.

In some embodiments, the crosslinked network of crosslinked polymer particles responds to an external stimulus like the polymers described previously. The external stimulus in such embodiments may be temperature or UV rays. In such situations, the shrinkage kinetics of the gel used in the compositions according to the present disclosure is asymmetric relative to the shrinkage kinetics of standard hydrogels: the rate of shrinkage of the gel is faster than that of a standard gel, whereas the rate of expansion of the gel (return to the initial state when it is no longer subjected to, e.g., heat) is not significantly higher than that of a standard gel.

The synthesis of the gels disclosed herein may be performed in two steps: (1) formation of crosslinked polymer particles, and then (2) assembly of the particles in a three-dimensional network, with formation of covalent bonds between the particles via the functional groups at their surface. This process of gel formation is described in patent application WO 03/022910.

The cosmetically active compound may be present in the composition in concentrations from 0.0001% to 60% by weight relative to the total weight of the composition. In some embodiments, the cosmetically active compound may be present in the composition in concentrations from 0.01% to 30% or from 0.1% to 15% by weight relative to the total weight of the composition.

The cosmetically acceptable medium for the present disclosure may comprise water or at least one cosmetically acceptable solvent or a mixture of water and at least one osmetically acceptable solvent. The cosmetically acceptable solvents may be chosen from, for example, alcohols, such as C₂-C₄ alcohols, for instance ethanol and isopropanol.

C₅-C₁₀ alkanes, such as acetone, methyl ethyl ketone, methyl acetate, butyl acetate, ethyl acetate, dimethoxyethane or diethoxyethane, may also be used as solvents. Fatty alcohols, modified or unmodified polyols, for instance glycerol, propylene glycol, or polyethylene glycols, volatile or non-volatile silicones, mineral, organic or plant oils, waxes and fatty acids, and mixtures thereof, may also be used as solvents herein.

In one embodiment, the cosmetically acceptable medium comprises water.

The compositions disclosed herein may be used for manufacturing numerous cosmetic products, including hair products such as products for fixing and/or holding the hair, hair conditioning products, such as sheen formulations, or hair care products.

These compositions may be packaged in various forms, such as pump-dispenser bottles or aerosol cans, so as to apply the composition in vaporized form or in mousse form. Such packaging forms may be used, for example, when it is desired to obtain a spray, a lacquer, or a mousse for fixing or treating the hair. These compositions may also be in the form of creams, gels, emulsions, lotions, or waxes.

The disclosed compositions may be in the form of a gel that can be used in cosmetics for making up the face and/or the body. In some embodiments, this gel may constitute a foundation or a makeup rouge, also known as a blusher.

One aspect of the disclosure relates to a makeup process that comprises applying a gel to the face. Water-based gels containing a hydrophilic phase comprising gelling agents formed from hydrophilic polymers are occasionally used for making up the skin. These water-based gels do not contain a fatty phase. They may also be colored by incorporating hydrophilic pigments and/or dyes, and may appear opaque or translucent. These gels may serve for the preparation of foundations or blushers. Translucent color water-based gels are known for imparting a “healthy complexion” and for their freshness on application; the user of such gels does not have the impression of wearing makeup.

But these gels may cause discomfort to individuals with dry skin, because of the absence of fatty substances, and also may cause a tacky and tensioning effect on the skin, due to the presence of gelling agents. In addition, these gels are often difficult to apply due to their lack of slipperiness and their excessively rapid drying, also these gels give the skin a heterogeneous makeup result, i.e., light and dark areas. To remedy the discomfort and drawbacks of these gels, moisturizers may be incorporated in these gels. Such moisturizers include polyols such as glycerol, but in this case, the tacky effect imparted to the skin is accentuated. These drawbacks make the use of conventional gels in cosmetic products difficult, or even unacceptable. But the gels comprising a composition as disclosed herein are, in contrast, easy to apply, slippery, spread satisfactorily, non-tacky, do not dry too quickly, and give the skin a uniform makeup result. They are also of particularly thick consistency, stable, non-lumpy, have a pleasant feel, and are compatible with electrolytes and/or primary alcohols.

When the compositions of the disclosure are packaged in aerosol form in order to obtain a lacquer or a mousse, the compositions comprise at least one propellant that may be chosen from volatile hydrocarbons such as n-butane, propane, isobutane and pentane, a halogenated hydrocarbon, and mixtures thereof. Carbon dioxide, nitrous oxide, dimethyl ether (DME), nitrogen, or compressed air may also be used as propellant. Moreover, mixtures of propellants may also be used. In one embodiment, dimethyl ether is used.

When used, the propellant is present in a concentration of from 5% to 90% by weight and, in some embodiments, a concentration of from 10% to 60% by weight relative to the total weight of the composition in the aerosol device.

A further aspect of this disclosure is a cosmetic gel comprising the cosmetic compositions disclosed above.

Finally, other aspects of the disclosure include a method of making a cosmetic composition, such as a hair or make-up composition, by including in the composition a gel comprising at least one crosslinked network of crosslinked polymer particles.

The cosmetic compositions of the present disclosure may further be used for holding and/or shaping keratin fibers, such as the hair.

A further aspect of the disclosure is a cosmetic hair process comprising the application of a composition as disclosed herein to the hair.

In one embodiment of the process disclosed herein, the process also comprises, simultaneously with or consecutive to the application of the composition to the hair, exposure of the hair to heat.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

The examples that follow are intended to illustrate the invention and should not be construed in any way as limiting the invention where it is described in broader terms in the specification and the claims. Notwithstanding that the numerical ranges and parameters setting forth the broad scope are approximations, the numerical values set forth in the specific examples below are reported as precisely as possible. Any numerical value, however, inherently contains certain errors necessarily resulting from the standard deviation found in its respective testing measurements.

### EXAMPLES

### Example 1

Composition 1 (according to the invention)

| | |
|---|---|
| Microgel of N-isopropylacrylamide (NIPAM) | 95.0% |
| Fixing polymer (1) | 0.5% |
| Water | 4.5% |

(1) polymer sold under the name Fixate G100 by the company Noveon

Composition 2 (reference composition)

| Carbopol (1) | 0.3% |
|---|---|
| Fixing polymer (2) | 0.50%* |
| Water | 99.2% |
| *indicates active material | |

(1) polymer sold under the name Synthalen K by the company Noveon
(2) polymer sold under the name Fixate G100 by the company Noveon

The NIPAM microgel was obtained according to the following procedure: 1.62 g of N-isopropylacrylamide monomer+0.165 g of 3-aminopropylmethacrylamide (7%)+0.06 g of standard crosslinking agent, for instance, methylbisacrylamide. The mixture was heated to 53° C., 0.056 g of surfactant (DTAB) and 50 mg of potassium persulfite were added as an initiator, and the pH was adjusted to 5.4. The particles were formed after reaction for 2 hours. Then, the mixture was freed of the surfactant. The medium was then concentrated, after which the gel was crosslinked with the same crosslinking agent.

1 g of composition based on NIPAM microgel (composition 1) was applied to 2.5 g of European hair.

1 g of reference composition (composition 2) was applied to 2.5 g of European chestnut-brown hair of the same nature.

After a leave-in time of 2 minutes, the hair was dried with a hair dryer or under a drying hood, both of which imparted heat to the process.

The locks to which compositions 1 and 2 were applied were then subjected to repeated circular motions. By comparing the hold of the locks formed after one hour, visually for half a day, it was found that the composition according to the invention (composition 1) gave the locks better hold and better rigidity.

### Example 2

Composition 1 (according to the invention)

| | |
|---|---|
| Microgel of N-isopropylacrylamide (NIPAM) | 95.0% |
| Fixing polymer (1) | 0.5% |
| Water | 4.5% |

(1) polymer sold under the name Fixate G100 by the company Noveon

Composition 3 (reference composition)

| | |
|---|---|
| Non-crosslinked poly-N-isopropylacrylamide (1) | 0.3% |
| Fixing polymer (2) | 3.0% |
| Water | 96.7% |

(1) product obtained by emulsion free-radical polymerization as
described, for example, in the article N. C. Woodward, B. Z.
Chowdhury, M. J. Snowden, S. A. Leharne, P. C. Griffiths, and
A. L. Winnington, Langmuir, 19, 3202-3211 (2003)
(2) polymer sold under the name Fixate G100 by the company Noveon

1 g of composition based on NIPAM microgel (see Example 1 above for mode of synthesis) was applied to 2.5 g of European hair. 1 g of reference composition 3 was applied to 2.5 g of European chestnut-brown hair of the same nature. After a leave-in time of 2 minutes, the hair was dried with a hairdryer or under a drying hood.

The locks to which compositions 1 and 3 were previously applied were then subjected to repeated circular motions. By comparing the hold of the locks formed after one hour, visually for half a day, it was found that the composition according to the invention (composition 1) gave the locks better hold and better rigidity.

1. A cosmetic composition comprising, in a cosmetically acceptable medium, at least one cosmetically active compound and at least one gel comprising at least one crosslinked network of crosslinked polymer particles.

2. The composition of claim 1, wherein said composition is a hair composition.

3. The composition of claim 1, wherein the polymer that forms the crosslinked network of crosslinked polymer particles is a polymer that responds to an external stimulus.

4. The composition of claim 3, wherein the external stimulus is chosen from temperature and UV rays.

5. The composition of claim 1, wherein the at least one cosmetically active compound is chosen from reducing agents or antioxidants, oxidizing agents, silicones, softeners, antifoams, moisturizers, emollients, plasticizers, sunscreens, dyes, pigments, fillers, clays, nacres or nacreous agents, fragrances, peptizers, thickeners, preserving agents, fixing polymers, vitamins or provitamins, conditioners, cationic polymers, cationic surfactants, waxes, and proteins.

6. The composition of claim 5, wherein the reducing agents are chosen from NaBH₄, KBH₄, BH₃, NADPH, NADH, H₂, 9-BBN (9 borabicyclo[3.3.1]nonane), LiAlH₄, NaAlEt₂H₂, Na₂S₂O₃, diisoamylborane, ascorbic acid, and cyclic or aliphatic compounds containing the enediol —(OH)C═C(OH)— chemical group, its isomeric forms, and the salts and esters thereof, hydroquinone, NaBH(OAc)₃, formamidinesulfinic acid (FASA), N-phenylformadiminesulfinic acid, phosphines, NaBH₃CN, Na₂S₂O₄, BMS (bismercaptoethyl sulfone), DTT (dithiothreitol), DMH (N,N′-dimethyl-N,N′-bis(mercaptoacetyl)hydrazine), thiols, thiolates, citrates, sulfites, bisulfites, monopersulfates and bisulfates of alkali metals, enzymes of the protein disulfide isomerase or thioredoxine type, and sulfinates.

7. The composition of claim 5, wherein the oxidizing agents are chosen from aqueous hydrogen peroxide solution, bromates, and persalts.

8. The composition of claim 5, wherein the thickeners are chosen from guar gum, xanthan gum, spruce meal, cellulose, modified derivatives thereof, and synthetic polymers.

9. The composition of claim 5, wherein the sunscreens are chosen from anthranilates; cinnamic derivatives; dibenzoylmethane derivatives; salicylic derivatives; camphor derivatives; triazine derivatives, such as 1,3,5-triazine derivatives; benzophenone derivatives; β,β′-diphenylacrylate derivatives; benzotriazole derivatives; benzalmalonate derivatives; benzimidazole derivatives; imidazolines; bis-benzazolyl derivatives; p-aminobenzoic acid (PABA) derivatives; methylenebis(hydroxyphenylbenzotriazole) derivatives; screening polymers and screening silicones; α-alkylstyrene-based dimers; 4,4-diarylbutadienes; and mixtures thereof.

10. The composition of claim 5, wherein the dyes are oxidation dyes chosen from oxidation bases and/or couplers.

11. The composition of claim 10, wherein the oxidation bases are chosen from ortho- and para-phenylenediamines, double bases, ortho- and para-aminophenols and heterocyclic bases, and the acid-addition salts thereof.

12. The composition of claim 11, wherein the heterocyclic bases are chosen from pyridine derivatives, pyrimidine derivatives, and pyrazole derivatives, and the acid-addition salts thereof.

13. The composition of claim 10, wherein the couplers are chosen from meta-phenylenediamines, meta-aminophenols, meta-diphenols, napthols, and heterocyclic couplers, and the acid-addition salts thereof.

14. The composition of claim 5, wherein the dyes are direct dyes.

15. The composition of claim 5, wherein the pigments are chosen from titanium dioxide, zirconium oxide, zinc oxide or cerium oxide, iron oxide or chromium oxide, manganese violet, ultramarine blue, chromium hydrate, ferric blue, carbon black, pigments of D&C, and lakes based on cochineal carmine or on barium, strontium, calcium or aluminium.

16. The composition of claim 5, wherein the nacres or nacreous agents are chosen from mica coated with titanium or with bismuth oxychloride, titanium mica with iron oxides, titanium mica with ferric blue or chromium oxide, titanium mica with an organic pigment and nacreous pigments based on bismuth oxychloride, and insoluble organic ethers or esters.

17. The composition of claim 5, wherein the fixing polymers are chosen from cationic, anionic, amphoteric, and nonionic fixing polymers, and mixtures thereof.

18. The composition of claim 17, wherein the cationic fixing polymers are chosen from homopolymers or copolymers of acrylic or methacrylic esters or amides containing amine functions, cationic polysaccharides, quaternary copolymers of vinylpyrrolidone and of vinylimidazole, cationic grafted polymers, and chitosans, and salts thereof.

19. The composition of claim 17, wherein the anionic fixing polymers are chosen from homopolymers or copolymers of acrylic and methacrylic acid or salts thereof, copolymers of acrylic or methacrylic acid with a monoethylenic monomer, crotonic acid copolymers, monounsaturated C₄-C₈ carboxylic acid or anhydride copolymers, polyacrylamides containing carboxylate groups, homopolymers or copolymers containing sulfonic groups, anionic polyurethanes, and anionic grafted silicone polymers.

20. The composition of claim 17, wherein the amphoteric fixing polymers are chosen from copolymers containing acidic vinyl units and basic vinyl units, crosslinked and acylated polyamino amides, polymers containing zwitterionic units, chitosan-based polymers, modified (C₁-C₅)alkyl vinyl ether/maleic anhydride copolymers, amphoteric polyurethanes, and amphoteric grafted silicone polymers.

21. The composition of claim 17, wherein the nonionic fixing polymers are chosen from polyalkyloxazolines, vinyl acetate homopolymers and copolymers, acrylic ester homopolymers and copolymers, acrylonitrile copolymers, styrene homopolymers and copolymers, polyamides, vinyllactam homopolymers other than vinylpyrrolidone homopolymers, vinyllactam copolymers, nonionic polyurethanes, and nonionic grafted silicone polymers.

22. The composition of claim 5, wherein the conditioners are chosen from natural or synthetic oils, natural or synthetic waxes, cationic surfactants, and conditioning cationic polymers.

23. The composition of claim 1, wherein the cosmetically active compound is present in the composition in an amount ranging from 0.0001% to 60% by weight relative to the total weight of the composition.

24. The composition of claim 23, wherein the cosmetically active compound is present in the composition in an amount ranging from 0.01% to 30% by weight relative to the total weight of the composition.

25. The composition of claim 24, wherein the cosmetically active compound is present in the composition in an amount ranging from 0.1% to 15% by weight relative to the total weight of the composition.

26. The composition of claim 1, wherein the size of the crosslinked polymer particles ranges from 1 to 100 nm.

27. The composition of claim 26, wherein the size of the crosslinked polymer particles ranges from 1 to 50 nm.

28. The composition of claim 1, wherein the crosslinked network of crosslinked polymer particles comprises particles with a mean spacing from 50 to 1000 nm.

29. The composition of claim 28, wherein the crosslinked network of crosslinked polymer particles comprises particles with a mean spacing from 50 to 500 nm.

30. The composition of claim 1, wherein the crosslinked polymer particles are present in an amount ranging from 0.4% to 60% relative to the total weight of the composition.

31. The composition of claim 30, wherein the crosslinked polymer particles are present in an amount ranging from 2% to 20% relative to the total weight of the composition.

32. The composition of claim 1, wherein the cosmetically acceptable medium comprises water, at least one cosmetically acceptable solvent, or a mixture of water and of at least one cosmetically acceptable solvent.

33. The composition of claim 32, wherein the cosmetically acceptable medium comprises water.

34. The composition of claim 1, wherein said composition is in a form chosen from a cream, a gel, an emulsion, a lotion, and a wax.

35. A cosmetic gel composition comprising, in a cosmetically acceptable medium, at least one cosmetically active compound and a gel comprising at least one crosslinked network of crosslinked polymer particles.

36. A method of making a cosmetic composition comprising including in the composition at least one gel comprising at least one crosslinked network of crosslinked polymer particles.

37. The method of claim 36, wherein the cosmetic composition is a cosmetic hair composition.

38. The method of claim 36, wherein the cosmetic composition is a cosmetic makeup composition.

39. A method for holding and/or shaping keratin fibers comprising applying to the keratin fibers a cosmetic composition comprising, in a cosmetically acceptable medium, at least one cosmetically active compound and at least one gel comprising at least one crosslinked network of crosslinked polymer particles.

40. The method of claim 39, wherein the keratin fibers are hair.

41. The method of claim 39, additionally comprising simultaneously with or consecutive to the application of the cosmetic composition to the keratin fibers, exposing the keratin fibers to heat.
